(19) United States
(12) Patent Application Publication
(10) Pub. No.: US 2002/0193424 A1
(43) Pub. Date: Dec. 19, 2002

(54) Human cancerous cell anti-proliferation and survival inhibition agent

(76) Inventor: Thomas J. Slaga, Austin, TX (US)

Correspondence Address: DAVID G. HENRY, P.O. Box 1470, Waco, TX, 76701, US

(21) Appl. No.: 10/155,843

(22) Filed: May 24, 2002

Related U.S. Application Data
(63) Continuation-in-part of application No. 09/533,142, filed on Mar. 23, 2000, now abandoned.

(51) Int. Cl. A61K 31/343; C07D 319/14

(52) U.S. Cl. 514/452; 549/362

(57) ABSTRACT

Silibinin derivatives useful in investigation of cancer control and prophylaxis and in the treatment and prophylaxis of cancers in humans.

### BACKGROUND OF THE INVENTION

1. Field of The Invention

Applicant's invention relates to chemical agents which are useful in arresting the proliferation of cancerous cells, human prostate cancer cells in particular.

2. Background Information

Prostate cancer (PCA) is the most common malignancy other than superficial skin cancers, and the second leading cause of cancer deaths in males in the USA (1). In 1997 alone, more than 334,500 new PCA cases were diagnosed with 41,800 attributable deaths (1). Clinical PCA incidence is low in Asian men, and highest in African-Americans and Scandinavians (2, 3). However, once moved to the United States, the incidence and mortality rate due to PCA are increased in Asian men approximating to those of Americans (3). The epidemiological studies suggest that dietary and environmental factors are the major cause for an increase in PCA in the United States men as well as in migrating Asians (2, 3). It is known that a low-fat and/or high-fiber diet significantly affects sex hormone metabolism in men (4). In Japan and some other Asian countries, despite the same incidence of latent small or non-infiltrating prostatic carcinomas, the mortality rate is low (3). This could, at least partly, be explained by a diet-related lowering of biologically active androgen (4). The importance of androgen in prostate carcinogenesis is also suggested by the observations that PCA rarely occurs in eunuchs or men with a deficiency in 5α-reductase, the enzyme responsible for converting testosterone to its active metabolite 5α-dihydrotestosterone (DHT) (5 and references therein). In addition, at least 75% of PCA with metastatic potential are androgen-dependent at initial diagnosis (6).

Androgen receptors (ARs) are required for the development of both normal prostate and PCA (7). A high proportion of mutations have been demonstrated in the ligand binding domain of the AR in hormone-refractory and metastatic PCA (7), and the mutant ARs including those described in the human prostate carcinoma LNCaP cells could be activated by estrogen and progesterone (7). Change in the specificity of the AR may provide a selective advantage in metastatic androgen independent PCA because they remain active after androgen ablation (7). A notable gene regulated by androgen in normal prostate as well as prostate carcinoma cells is prostate-specific antigen (PSA) (8). PSA has been demonstrated to be a sensitive and specific tumor marker for PCA screening and assessment (9), and is used as an indicator of disease and response to therapy in a number of trials of cytotoxic agents in PCA (10). Several trials have also shown a direct relationship between decline in PSA and shrinkage of bi-dimensionally measurable prostate tumor (11). Whereas stimulation of the mutant AR in LNCaP cells by androgen does not differ from stimulation of the wild-type AR, the estrogenic substance and some anti-androgens bind to this receptor with higher affinity, efficiently stimulate its transactivation function, and increase PSA levels in the supernatant of LNCaP cells (7).

Traditional Asian diets, and to a lesser extent those of Mediterranean people as well as vegetarians, are not only low in animal proteins and fat, and high in starch and fiber, they are also rich in ‘weak plant estrogens’ (12, 13). These phytoestrogens are excreted in large amounts in the urine and serum (12, 13). Some of these phytoestrogens possess weak estrogenic, anti-estrogenic and antioxidant activity, and therefore possess the potential for exerting an influence on hormone-dependent cancers including PCA (12, 13). In recent years, two groups of phytoestrogens, namely polyphenolic antioxidants (e.g. isoflavonoids, flavonoids, etc) and lignans are receiving increasing attention for the prevention and/or intervention of human malignancies including PCA (12-14). Silymarin, a polyphenolic flavonoid antioxidant isolated from milk thistle, has human acceptance as it is used clinically in Europe and Asia for the treatment of alcoholic liver diseases; as a therapeutic agent, it is well tolerated and largely free of adverse effects (15, 16). So much so, silymarin is being marketed recently in the United States and Europe as nutritional supplement by several Phytochemical/Nutraceutical Companies. In studies utilizing the mouse epithelial models of carcinogenesis, recently we have shown that silymarin affords exceptionally high to complete protection against experimental tumorigenesis (15, 16). Likewise, experiments involving a mammary gland culture initiation-promotion protocol also demonstrated the ability of silymarin to inhibit tumor promotion (17). Based on structural similarity of silymarin with phytoestrogens in terms of a polyphenolic flavonoid skeleton, and its strong anti-carcinogenic effects in different tumorigenesis studies, the present inventors in prior studies reasoned that silymarin could also be a useful agent for the intervention of human PCA. It was shown that silymarin indeed decreases the intracellular and secreted levels of PSA protein in human prostate carcinoma LNCaP cells under both serum- and androgen-stimulated conditions concomitant with the inhibition of cell growth via G1 arrest in cell cycle progression. The G1 arrest by silymarin does not lead to apoptosis but causes neuroendocrine differentiation of the cells.

Despite the promising results in attacking human prostate cells and intervening in their proliferation through the use of silymarin, and short of a absolute curative “silver bullet” for human PCA, there remains, and always will remain, a compelling need for ever better, more potent agents to attack human PCA cells. Even if uses of such agents (when discovered or derived) are, at least initially, in vitro, utility still exists therefor as they provide instructive foundations upon which in vivo uses of the agents themselves, or of derivations thereof in the combating of cancers, PCA and otherwise.

### SUMMARY OF THE INVENTION

In view of the foregoing, it is an object of the present invention to provide a novel and useful chemical entity.

It is another object of the present invention to provide a chemical agent which is useful in arresting proliferation of cancer cells.

It is another object of the present invention to provide a chemical prophylaxis applicable to human cancer cell genesis or proliferation.

It is another object of the present invention to provide a chemical agent which is a derivative of a substrate known to have anti-cancer properties, but which, dose for dose, is more effective than such substrate in arresting cancer cell proliferation and/or effecting increased rates of cell death than the substrate.

In satisfaction of the above objects and more specific subsets thereof, the present inventors provide and herein teach derivatives of silibinin (regarded as the primary active ingredient of silymarin) which, laboratory evidence establishes, is substantially more efficacious in arresting tumor growth than silymarin/silibinin itself.

The presently claimed compounds are useful in vivo as agents for attacking PCA tumors and providing means for deriving useful information on PCA prophylaxis and treatment, and is predictably safe and efficacious in preventing and treating PCA in vivo, in part, because silymarin is already used in treating certain human cancer and is of know human tolerance and non-toxicity.

### DESCRIPTION OF THE FIGURES

FIG. 1 depicts the chemical reaction in which silibinin is converted into Derivative #1 of the present invention (2,3-Dihydro-3-[3,4-dimethoxyphenyl]-2-[hydroxymethyl]-5,7-dimethoxy-4-oxobenzopyran-2-yl]benzodioxin).

FIG. 2 depicts the chemical reaction in which Derivative #1 of the present invention (2,3-Dihydro-3-[3,4-dimethoxyphenyl]-2-[hydroxymethyl]-5,7-dimethoxy-4-oxobenzopyran-2-yl]benzodioxin) is converted into Derivative #2 of the present invention (2-3-Dihydrogensuccinate of 2,3-Dihydro-3-[3,4-dimethoxyphenyl]-2-[hydroxymethyl]-5,7-dimethoxy-4-oxobenzopyran-2-yl]benzodioxin).

FIG. 3 is a graphical depiction of the comparative growth curves of DU145 cells upon treatment with varying doses of Silymarin and Derivatives #1 and #2 of the present invention, measured at 48 and 72 hours.

FIG. 4 depicts the numerical data upon which the graphical depiction of FIG. 3 is based.

FIG. 5 is a graphical depiction of the comparative percent cell death rates of DU145 cells upon treatment with varying doses of Silymarin and Derivatives #1 and #2 of the present invention, measured at 48 and 72 hours.

FIG. 6 depicts the numerical data upon which the graphical depiction of FIG. 5 is based.

### DETAILED DESCRIPTION OF INVENTIVE WORK

A. SYNTHESIS OF SILIBININ DERIVATIVES 1 and 2.

Two novel derivatives of silibinin have been formulated by the present inventors and have shown significantly increased efficacy in countering the survival and proliferation of human prostate cancer cells. The steps in their synthesis will now be described.

¹NMR (400 MHz) and ¹³C (100 MHz) were recorded on a JEOL eclipse series 400 MHz spectrometer using CDCl₃ or DMSO d₆ as an internal standard. Mass spectra were taken with a Fisons-VG platform LC/MS. Commercially available reagents and HPLC grade or anhydrous solvents were used without further purification. Normal phase column chromatography and TLC were performed on ICN silica gel (63-200, 60A) and Merck silica gel (60A) with fluorescent indicator respectively.

1. 2,3-Dihydro-3-[3,4-dimethoxyphenyl]-2-[hydroxymethyl]-5,7-dimethoxy-4-oxobenzopyran-2-yl]benzodioxin (Derivative 1)

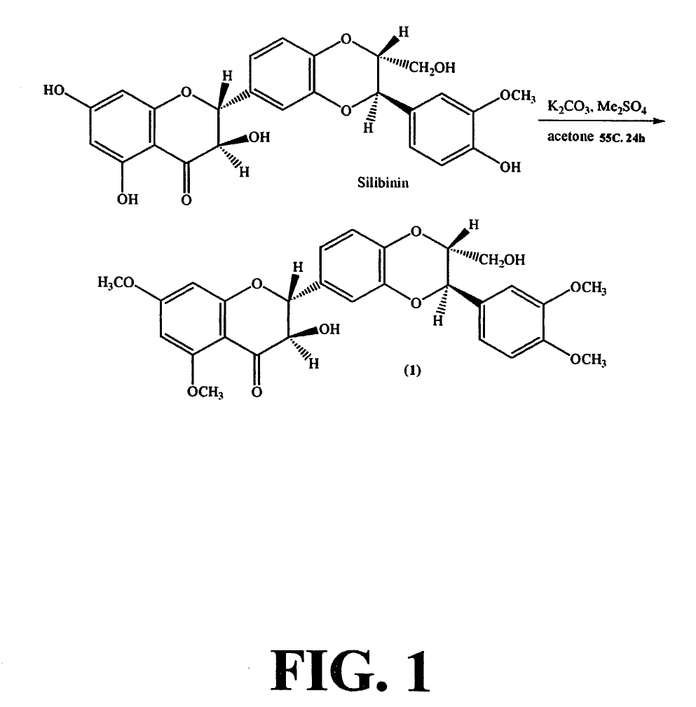

Refer to FIG. 1 for a depiction of the structures of the initial substrate (silibinin) and the resulting first derivative of the present invention (2,3-Dihydro-3-[3,4-dimethoxyphenyl]-2-[hydroxymethyl]-5,7-dimethoxy-4-oxobenzopyran-2-yl]benzodioxin).

The synthesis of 2,3-Dihydro-3-[3,4-dimethoxyphenyl]-2-[hydroxymethyl]-5,7-dimethoxy-4-oxobenzopyran-2-yl]benzodioxin proceeds as follows: To a solution containing 1.00 g (2.07 mmol) of silibinin in 35 mL of acetone under an atmosphere of nitrogen, 1.8 mL (19.02 mmol) of dimethyl sulfate followed by 1.00 g (7.24 mmol) of potassium carbonate. The reaction was heated to 50° C. and allowed to stir for 24 h while monitoring by TLC (80% ethyl acetate/hexane). The reaction was diluted with and washed with water. The organic phase was dried over magnesium sulfate. Filtration, removal of solvent under reduced pressure and column chromatography of the residue on silica gel eluting with 80% ethyl acetate/hexane gave 0.85 g (78.29%) of produce as a white solid.

¹H NMR (DMSO d₆) δ3.32-3.38 (m, 1H), 3.54-3.58 (m, 1H), [3.78 (s), 3.80 (s), 12H], 4.18-4.23 (m, 1H), 4.36-4.42 (m, 1H), 4.97-5.00 (m, 2H), 5.05 (d, J=11.3 Hz, 1H), 5.36 (s(broad), 1H), 6.17-6.18 (m, 1H), 6.22-6.23 (m, 1H), 6.97-7.11 (m, 6H); ¹H NMR (DMSO d₆ with D₂O) δ3.34 (dd, J=4.7, 12.1 Hz, 1H0, 3.53-3.58 (m, 1H), [3.76(s), 12H], 4.16-4.22 (m, 1H), 4.36 (dd, J=4.3, 11.0 Hz, 1H), 4.95 (d, J=7.8 Hz, 1H), 5.03 (d, J=11.3 Hz, 1H), 6.15 (bs, 1H), 6.20 (bs, 1H), 6.96-7.07 (m, 6H); ¹³C NMR (DMSO d₆ with D₂O) δ56.01, 56.06, 56.29, 56.45, 60.56, 72.89, 76.22, 78.50, 82.67, 93.41, 94.05, 104.01, 111.57, 112.04, 116.94, 120.79, 121.57, 121.76, 129.49, 130.70, 143.64, 144.05, 149.26, 149.61, 162.19, 164.26, 166.29, 190.71; MS Calculated 524; Found 525 (M+H), 547 (M+Na).

2. 2-3-Dihydrogensuccinate of 2,3-dihydro-3-[3,4-dimethoxyphenyl]-2-[hydroxymethyl]-6-[3-hydroxy-5,7-dimethoxy-4-oxobenzopyran-2-yl]benzodioxin (Derivative 2)

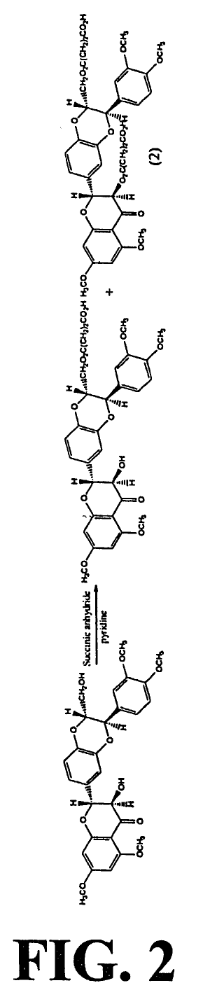

Refer to FIG. 2 for a depiction of the structures of the initial substrate (2,3-dihydro-3-[3,4-dimethoxyphenyl]-2-[hydroxymethyl]-6-[3-hydroxy-5,7-dimethoxy-4-oxobenzopyran-2-yl]benzodioxin) and the resulting second derivative of the present invention (2-3-Dihydrogensuccinate of 2,3-Dihydro-3-[3,4-dimethoxyphenyl]-2-[hydroxymethyl]-5,7-dimethoxy-4-oxobenzopyran-2-yl]benzodioxin).

The synthesis of the 2-3-Dihydrogensuccinate of 2,3-Dihydro-3-[3,4-dimethoxyphenyl]-2-[hydroxymethyl]-5,7-dimethoxy-4-oxobenzopyran-2-yl]benzodioxin of the present invention proceeds as follows:

To a solution containing 1.00 g (1.91 mmol) of 2,3-dihydro-3-[3,4-dimethoxyphenyl]-2-[hydroxymethyl]-6-[3-hydroxy-5,7-dimethoxy-4-oxobenzopyran-2-yl]benzodioxin in 8 ml of pyridin was added 0.42 g (4.20 mmol) of succinic anhydride under a nitrogen atmosphere. The reaction was heated to 45° C. for 24 h. An aliquot was drawn, diluted with ethyl acetate and washed with 1N hydrochloric acid. TLC of the organic phase indicated mono and bis-succinate derivatives (10% methanol/dichloromethane). An additional 0.21 g (2.10 mmol) of succinic anhydride added and stirring continued for 24 h. The reaction was diluted with acetate and washed with 1N hydrochloric acid. The organic phase was washed with brine and water and dried of sodium sulfate. Filtration and removal of solvent gave 1.26 g of a pale yellow solid. Column chromatography of the residual solid on silica gel eluting with 10-15% methanol/chloroform gave 0.598 g (43.21%) of product.

¹H NMR (DMSO d₆) δ2.10-2.55 (m, 8H), [3.76 (s), 3.78 (s), 3.80 (s), 12H], 3.93 (bd, J=8.6 Hz, 1H), 4.08-4.13 (m, 1H), 4.54 (d, J=5.8 Hz, 1H), 5.00 (d, J=6.3 Hz, 1H) 5.44 (d, J=10.9 Hz, 1H), 5.62 (dd, J=11.7, 19.5 Hz, 1H), 6.21 (s, 1H), 2.23 (s, 1H), 6.98-7.17 (m, 6H); ¹³C NMR (DMSO d₆) δ30.17, 31.07, 51.77, 56.00, 56.09, 56.42, 56.53, 62.79, 73.86, 75.61, 75.89, 79.74, 80.02, 93.75, 94.33, 104.10, 111.82, 112.81, 116.54, 117.17, 120.75, 128.72, 129.55, 143.67, 143.91, 143.97, 149.31, 149.75, 162.38, 164.13, 166.58, 171.86, 172.79, 173.58, 184.50; MS Calcd.: 724; Found: 747 (M+Na), 763 (M+K)

B. ANALYSIS OF EFFICACY OF DERIVATIVES OF SILIBININ

1. Cells and Culture Conditions. Human prostate carcinoma DU145 cells were obtained from American Type Culture Collection (Bethesda, Md.), and cultured in RPMI 1640 containing 10% fetal bovine serum (FBS) and 1% penicillin-streptomycin under standard culture conditions. Alternatively, DU145 cells were cultured in 10% charcoal/dextran stripped FBS (cFBS) and 1% penicillin-streptomycin in the presence or absence of 1 nM DHT.

One object of the investigations which led to the present invention was to compare the efficacy of the subject Silibinin derivatives with Silymarin (already known to be of some efficacy in this context). Therefore, in addition to control treatments, Silymarin was used to treat the same cells as the Silibinin derivatives and the comparative results are depicted in the tables.

Silymarin and Silibinin derivatives were dissolved in ethanol for all the treatments. The final concentration of ethanol in culture medium during silibinin derivatives treatment did not exceed 0.5% (v/v), and, therefore, the same concentration of ethanol was added in control medium. The dose and time of silibinin derivatives treatment are depicted in the associated drawings.

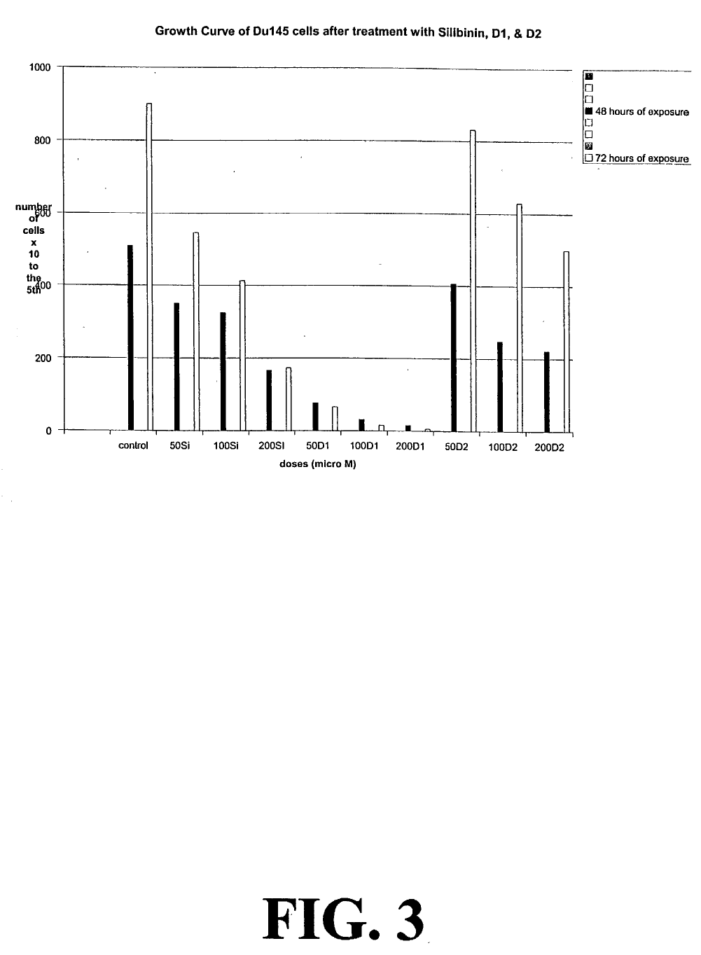

2. Results. Referring to FIGS. 3 through 6, for the studies assessing the effect of silibinin derivatives on FBS-stimulated cell growth and cell death, DU145 cells were plated at 1×10⁵ cells per 60 mm plate in RPMI 1640 containing 10% FBS and treated with either ethanol alone or varying concentrations of silymarin (“Si”) and silibinin derivatives (“D1” and “D2”) (50, 100 and 200 micromolars). Cells were counted at 48 and 72 hours after treatment with the subject compounds.

At the counting intervals, cells were trypsinized and collected in counting vials. Each plate was washed thoroughly with isotonic buffer containing 0.1% formalin and washings were collected in the original vials with trypsinized cells. Each vial was counted using Coulter Counter to determine the total cell number.

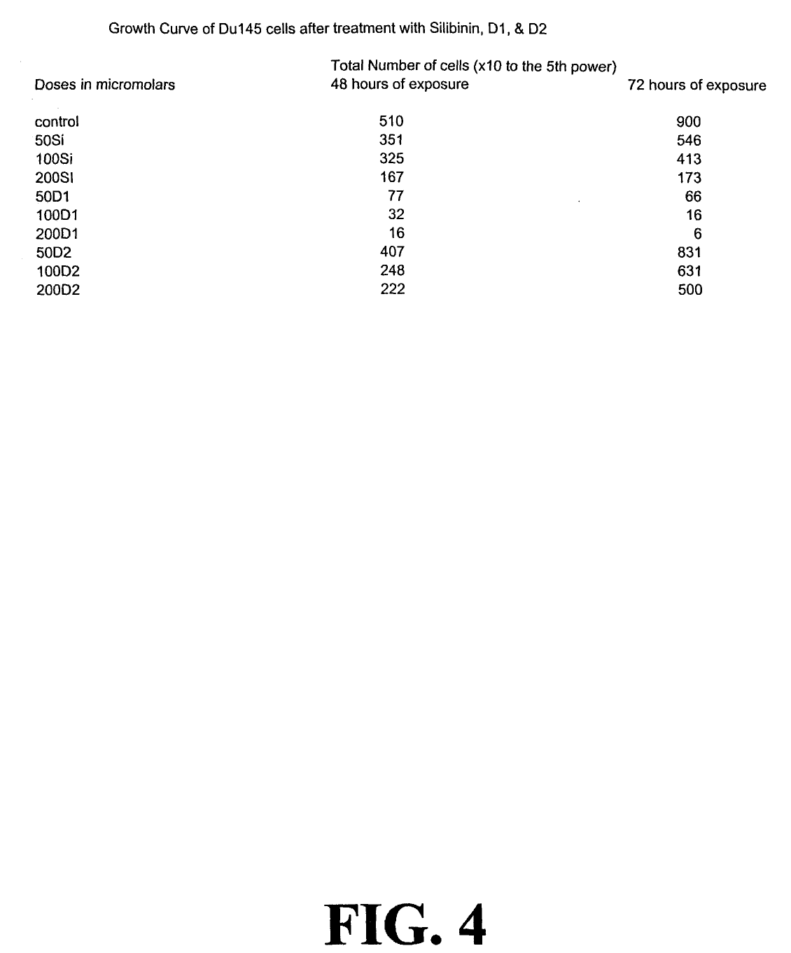

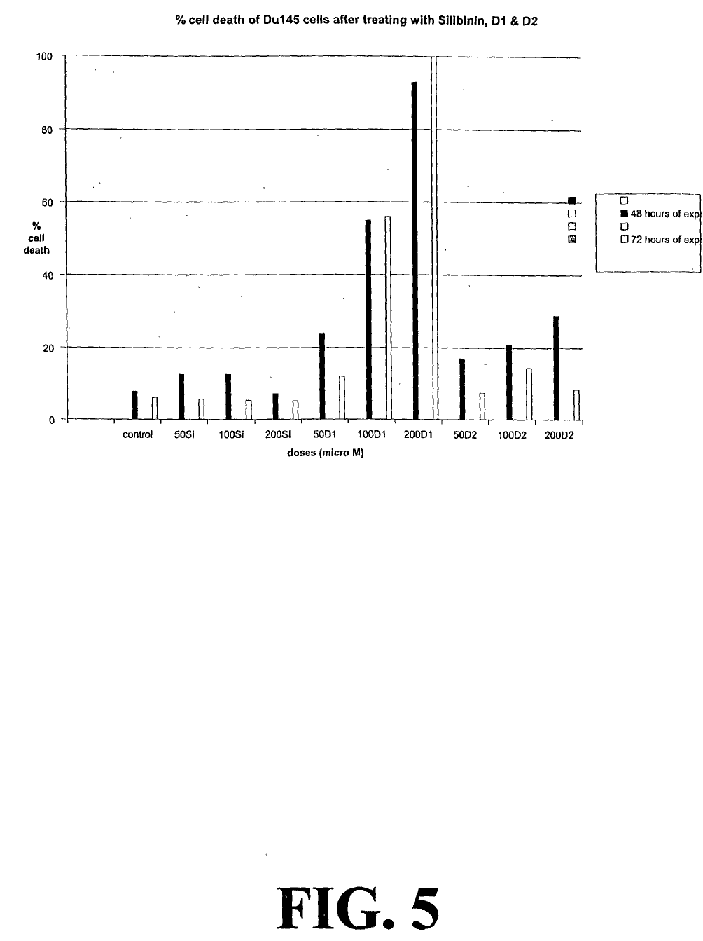

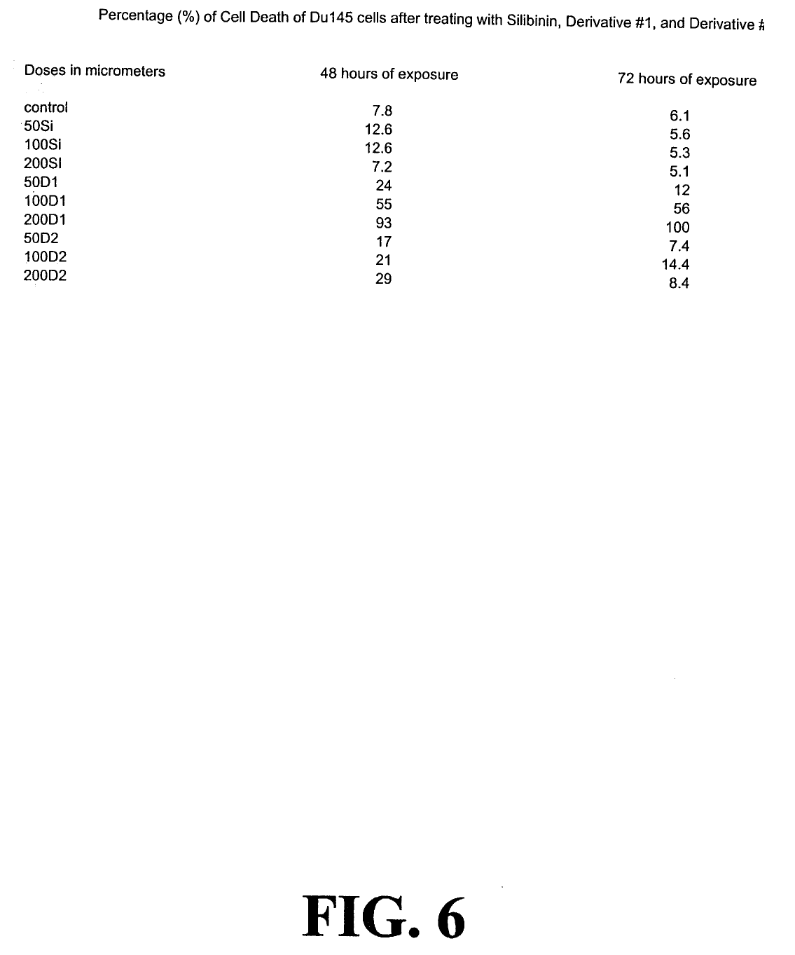

As is clear from the numeric data shown in FIGS. 4 and 6, and the graphical depiction of the data as shown in FIGS. 3 and 5, derivative #1 of the present invention (2,3-Dihydro-3-[3,4-dimethoxyphenyl]-2-[hydroxymethyl]-5,7-dimethoxy-4-oxobenzopyran-2-yl]benzodioxin) effects a remarkably improved inhibition of cell growth over the control and silymarin, and a likewise improved percentage of cell death.

In view of (1) the known efficacy of silymarin in the treatment and prophylaxis of cancers, (2) the application of the same in vitro analysis of silymarin in assessing such efficacy as that used in making such assessment for the present silibinin derivatives, and (3) the mechanism of action which accounts for the improved efficacy of the present derivatives, undue experimentation will not be required to achieve in vivo therapeutic effects.

3. Link Between Structural Changes in Compound and Increased Efficacy. The methoxy substitution in the present silibinin derivatives significantly increases the ability of the compound to inhibit growth of human prostate cancer cells. The succinate substitution, on the other hand, increases absorption of the compound into the circulatory system and also improves entrance into cells. The combination of the methoxy and succinate substitutions further increases the effectiveness of the modified silibinin structure to inhibit human prostate cancer cell growth.

The thrust of the work of the present inventors is that the present silibinin derivatives inhibit both serum- and androgen-stimulated PSA protein levels in DU145 cells concomitant with cell growth inhibition via a G1 arrest in cell cycle progression. The silibinin derivatives-treated DU145 cells unable to grow, follow a differentiation pathway as evidenced by neuroendocrine like morphology, elevated prostate tissue differentiation markers cytokeratins 8 and 18 and altered cell cycle regulatory molecules. Based on these findings, the conclusion is inescapable that the silibinin derivatives of the present invention will have utility as both an in vitro study compound and as an anti-proliferative differentiating agent for the intervention of hormone-refractory human prostate cancer in vivo.

4. Presently Anticipated Human Application of PCA Prevention and Intervention, and Best Mode of Application. The immediate as well as future applications of this invention include consumption of silibinin derivatives a) to reduce the risk of prostate cancer, and b) to intervene in existing cases of prostate cancer, as well as the in vitro use of the present, novel compounds in developing, perhaps, still more efficacious compounds, or proving new uses for the present compounds.

Whether for prevention or treatment of PCA, a present dosage suggestion (presently for clinically conducted and approved experimental purposes only) is a dosage, in pill form, of silibinin derivatives, with pharmacologically inert fillers and binding agents, at a rate of approximately 150 mg.-450 mg. active ingredient per day for an approximately 70 kg human male. Such daily consumption would, at present contemplation, be taken via three or four pills per day at substantially equidistance time intervals throughout each day. It is expected, though not yet clinically confirmed, that a lower dosage would likely be efficacious in the prevention of PCA, than would be required for treatment of existing PCA.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

1. Wingo, P. A., Landis, S. & Ries, L. A. G. (1997) CA Cancer J. Clin. 47, 239-242.

2. Parker, S. L., Tong, T., Bolden, S. & Wingo, P. A. (1997) CA Cancer J. Clin. 47, 5-27.

3. Shimizu, H., Ross, R. K., Bernstein, L., Yatani, R., Henderson, B. E. & Mack, T. M. (1991) Br. J. Cancer 63, 963-966.

4. Anderson, K. E., Rosner, W., Khan, M. S., New, M. I., Pang, S., Wissel, P. S. & Kappas, A. (1987) Life Sci. 40, 1761-1768.

5. Aquilina, H. W., Lipsky, J. J. & Bostwick, D. G. (1997) J. Natl. Cancer Inst. 89, 689-696.

6. Thompson, I. M., Coltman, C. A., Brawley, O. W. & Ryan, A. (1995) Semin. Urol. 13, 122-129.

7. Culig, Z., Hobisch, A., Hittmair, A., Peterziel, H., Cato, A. C. B., Bartsch, G. & Klocker, H. (1998) The Prostate 35, 63-70.

8. Wang, L. G., Liu, X. M., Kreis, W. & Budman, D. R. (1997) Cancer Res. 57, 714-719.

9. Stamey, T. A. & Kabalin, J. N. (1989) J. UroI. 141, 1070-1075.

10. Cadeddu, J. A., Pearson, J. D., Partin, A. W., Epstein, J. I. & Carter, H. B. (1993) Urology 42, 383-389.

11. Brausi, M., Jones, W. G., Fossa, S. D., de Mulder, P. H., Droz, J. P., Lentz, M. A., van Glabbeke, M. & Pawinski, A. (1995) Eur. J. Cancer 31A, 1622-1626.

12. Adlercreutz, H., Fotsis, T., Bannwart, C., Wahala, K., Makela, T., Brunow, G. & Hase, T. (1986) J. Steroid Biochem. 25, 791-797.

13. Morton, M. S., Chan, P. S. F., Cheng, C., Blacklock, N., Matos-Ferreira, A., Abranches-Monteiro, L., Correia, R., Lloyd, S. & Grfffiths, K. (1997) The Prostate 32, 122-128.

14. Sun, X.-Y., Plouzek, C. A., Henry, J. P., Wang, T. T. Y. & Phang, J. M. (1998) Cancer Res. 58, 2379-2384.

15. Katiyar, S. K., Korman, N. J., Mukhtar, H. & Agarwal, R. (1997) J. Natl. Cancer Inst. 89, 556-566.

16. Zi, X., Mukhtar, H. & Agarwal, R. (1997) Biochem. Biophys. Res. Commun. 239, 334-339.

17. Mehta, R. G. & Moon, R. C. (1991) Anticancer Res. 11, 593-596.

18. Zi, X., Feyes, D. K. & Agarwal, R. (1998) Clin. Cancer Res. 4, 1055-1064.

19. Elledge, S. J. & Haper, J. W. (1994) Curr. Opin. Cell Biol. 6, 847-852.

20. Sherr, C. J. (1994) Cell 79, 551-555.

21. White, E. (1995) Genes Dev. 10, 1-15.

22. Piette, J., Neel, H. & Marechai, V. (1997) Oncogene 15, 1001-1010.

23. Hsieh, T. C., Xu, W. & Chiao, J. W. (1995) Expt. Cell Res. 218, 137-143.

24. Lee, C., Sutkowski, D. M., Sensibar, J. A., Zelner, D., Kim, I., Amsel, I., Shaw, N., Prins, G. S. & Kozlowski, J. M. (1995) Endocrinology 136, 796-803.

25. Thalmann, G. N., Anezinis, P. E., Chang, S.-M., Zhau, H. E., Kim, E. E., Hopwood, V. L., Pathak, S., von Eschenbach, A. C. & Chung, L. W. K. (1994) Cancer Res. 54, 2577-2581.

26. Ban, Y., Wang, M. C., Watt, K. W. K., Loor, R. & Chu, T. M. (1984) Biochem. Biophys. Res. Commun. 123, 482-488.

27. Lijia, H. A. A. (1985) J. Clin. Invest. 76, 1899-1903.

28. Lu, S, Tsai, S. Y. & Tsai, M.-J. (1997) Cancer Res. 57, 4511-4516.

29. Quelle, D. E., Ashmun, R. A., Shuttleff, S. A., Kato, J.-Y, Bar-Sagi, D., Roussel, M. F. & Sherr, C. J. (1993) Genes Dev. 7, 1559-1571.

30. Tam, S. W., Theodoras, A. M., Shay, J. W., Draetta, G. F. & Pagano, M. (1994) Oncogene 9, 2663-2674.

31. Buckley, M. F., Sweeney, K. J. E., Hamilton, J. A., Sini, R. L., Manning, D. L., Nicholson, R. I., deFazio, A., Watts, C. K. W., Musgrove, E. A. & Sutherland, R. L. (1993) Oncogene 8, 2127-2133.

32. Lovec, H., Sewing, A., Lucibello, F. C., Muller, R. & Moroy, T. (1994) Oncogene 9, 323-326.

33. Mueller, A., Odze, R., Jenkins, T. D., Shahsesfaei, A., Nakagawa, H., Inomoto, T. & Rustgi, A. K. (1997) Cancer Res. 57, 5542-5549.

34. Chen, Y., Navone, N. M. & Conti, C. J. (1995) Urol. Oncol. 1, 101-108.

35. Chen, Y., Robles, A. I., Martinez, L. A., Liu, F., Gimenez-Conti, I. B. & Conti, C. J. (1996) Cell Growth Differ. 7, 1571-1578.

36. Chen, Y., Martinez, L. A., Lacava, M., Coghlan, L. & Conti, C. J. (1998) Oncogene 16, 1913-1920.

37. Zi, X., Grasso, A. W., Kung, H.-J. & Agarwal, R. (1998) Cancer Res. 58, 1920-1929.

38. Clurman, B. E. & Roberts, J. M. (1995) J. Natl. Cancer Inst. 87, 1449-1501.

39. Guo, K., Wang, J., Andres, V., Smith, R. C. & Walsh, K. (1995) Mol. Cell. Biol. 15, 3823-3829.

40. Missero, C., Calautti, E., Eckner, R., Chin, J., Tsai, L. H., Livingston, D. M. & Dotto G. P. (1995) Proc. Natl. Acad. Sci. USA 92, 5451-5455.

41. Jiang, H., Lin, J., Su, Z., Herlyn, M., Kerbel, R. S., Weissman, B. E, Welch, D. R. & Fisher, P. B. (1995) Oncogene 10, 1855-1864.

42. Hollstein, M., Sidransky, D., Vogelstein, B. & Harris, C. C. (1991) Science 253, 49-53.

43. Dulic, V., Kaufmann, W. K., Wilson, S. J., Tlsty, T. D., Lees, E., Harper, J. W., Elledge, S. J. & Reed, S. I. (1994) Cell 76, 1013-1023.

44. Reynisdottir, I., Polyak, K., Iavarone, A. & Massague, J. (1995) Genes Dev. 9, 1831-1845.

45. Poluha, W., Poluha, D. K., Chang, B., Crosbie, N. E., Schonhoff, C. M., Kilpatrick, D. L. & Ross, A. H. (1996) Mol. Cell. Biol. 16, 1335-1341.

46. Hengst, L. & Reed, S. I. (1996) Science 271, 1861-1864.

47. Haupt, Y., Maya, R., Kazaz, A. & Oren, M. (1997) Nature 387, 296-299.

48. Dazard, J.-E., Auglas, D., Neel, II., Mils, V., Marechal, V., Basset-Seguin, N. & Piette, J. (1997) Oncogene 14, 1123-1128.

49. Mayo, L. D. & Berberich, S. J. (1996) Oncogene 13, 2315-2321.

I claim:

1. A chemical compound of the following structure:

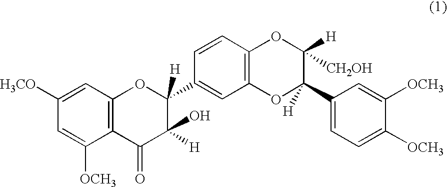

2. A therapeutic agent for use in inhibiting the proliferation of human cancer cells, such agent, being derived from silibinin, and having substantially the following structure:

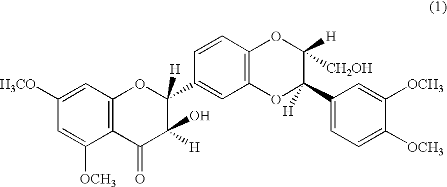

3. A chemical agent for in vitro application in the inhibition of cancer cell growth and survival, such agent having the following structure:

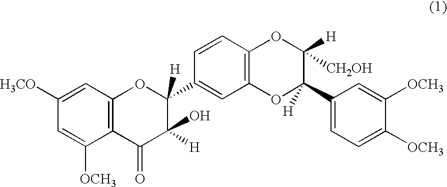

4. A method for preventing and treating prostate cancer comprising the steps of:
selecting a dosage form comprising a silibinin derivative of the following structure: 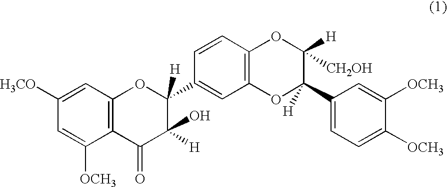
administering a plurality of said dosage form to human recipients at a rate whereby said recipient receives between approximately 150 mg and 450 mg of silibinin derivatives per day for a plurality of days.
